(19) United States
(12) Patent Application Publication
(10) Pub. No.: US 2022/0125326 A1
(43) Pub. Date: Apr. 28, 2022

(54) SYSTEM AND METHOD FOR IMAGING AND SEGMENTATION OF CAVERNOUS NERVES

(71) Applicant: ROSTAK LLC, Brooklyn, NY (US)

(72) Inventors: Ilya PYATNITSKIY, Krasnogorsk (RU); Oleg VORONTSOV, Moscow (RU); Roman OVCHINNIKOV, Moscow (RU)

(21) Appl. No.: 16/949,265

(22) Filed: Oct. 22, 2020

(51) Int. Cl. A61B 5/026; A61B 5/055; A61B 5/00; G01R 33/56; G01R 33/50

(52) U.S. Cl. CPC A61B 5/0263; A61B 5/0555; G01R 33/50; A61B 5/004; G01R 33/56; A61B 5/0037

(57) ABSTRACT

A system and a method for improved imaging and segmentation of cavernous nerves are disclosed herein. Some exemplary embodiments are related to a method of imaging erectile nerves using a magnetic resonance imaging (MRI) system. The method includes positioning a first radiofrequency (RF) coil on a first side of a body of a patient disposed on an MRI table and a second RF coil on a second side of the body of the patient symmetrical with respect to the first RF coil; performing a localizer scan sequence to determine plotting of image slices; performing a T2-weighted true (FISP) cine MRI sequence of the pelvic region to determine whether patient physiological activity corresponds to a desired condition; generating a plurality of images of the pelvic region using an MRI sequence protocol; and performing post-processing and 3D reconstruction on the plurality of images to map the at least one erectile nerve.

### BACKGROUND

Magnetic resonance imaging (MRI) is an imaging modality that distinguishes objects based on their composition. MRIs are capable of providing both 2-dimensional and 3-dimensional images. An MRI system typically includes a primary magnet that provides a static magnetic field, magnetic field gradient coils and radio frequency (RF) coils. The primary magnet generally provides a homogeneous magnetic field within a space within which the patient is placed.

The uniform magnetic field generated by the main magnet is applied to an imaged object along the Z-axis of a Cartesian coordinate system, the origin of which is within the imaged object. The uniform magnetic field aligns the magnetization arising from the nuclei of the atoms of the imaged object along the Z-axis. RF magnetic field pulses of a selected frequency, with field direction orientated within the XY plane, cause the nuclei to resonate at their Larmor frequencies. In a typical planar imaging sequence, an RF signal centered about the desired Larmor frequency is applied to the imaged object at the same time at which a magnetic field gradient is applied along the Z-axis. This gradient field excites into resonance the nuclei of only those atoms in a slice having a defined thickness through the object perpendicular to the Z-axis.

After excitation of the nuclei of the slice, magnetic field gradients are applied along the X and Y axes respectively. The gradient along the X axis causes the nuclei in the slice to precess at different frequencies depending on their positions along the X axis. Thus, this gradient is often referred to as a frequency encoding or read-out gradient. The Y axis gradient is incremented through a series of values and encodes the Y position into the rate of change of the phase of the precessing nuclei as a function of gradient amplitude, which is referred to as phase encoding. Two basic parameters of an MRI system are echo time (TE) and repetition time (TR). The parameters are typically measured in milliseconds (ms). TE represents the time from the center of the RF-pulse to the center of the echo. For pulse sequences with multiple echoes between each RF pulse, several echo times may be defined and are commonly referred to as TE1, TE2, TE3, etc. TR is the length of time between corresponding consecutive points on a repeating series of pulses and echoes.

The quality of the image produced by the MRI is dependent, in part, upon the strength of the MR signal received from the precessing nuclei. For this reason, an independent RF coil is often placed in close proximity to the region of interest of the imaged object (i.e., on the surface of the imaged object) to improve the strength of the received signal. Such RF coils are sometimes referred to as local or surface coils.

The strength of an MRI system is typically defined in terms of its magnetic flux density; or Tesla. Three popular MRI systems are the 1.5 Tesla (or 1.5 T), the 3.0 T, and the 7.0 T systems. The 7.0 T is typically used in research settings and, while it will provide the most detailed images of the three, is not typically found in clinical settings due to its extremely high cost. 1.5 T MRI systems are the most commonly used systems for. However, the increased magnet strength of a 3.0 T MRI system is preferable in some cases such as, for example, neuroimaging or MR-angiography studies. The 3.0 T MRI system provides an improved signal-to-noise ratio (SNR) compared to the 1.5 T MRI system. However, 3.0 T MRI images are more likely to generate artifacts caused by noise. The 1.5 T MRI requires longer scans to create clear images, while the 3.0 T MRI system takes a shorter amount of time due to the increased signal strength.

The two basic types of MRI images are T1-weighted images and T2-weighted images. The timing of radiofrequency pulse sequences used to make T1 images results in images which highlight fat tissue within the body. The timing of radiofrequency pulse sequences used to make T2 images results in images which highlight fat and water within the body.

Most MRI systems have developed an extensive list of imaging protocols for various diseases and clinical scenarios. Each protocol typically contains numbers of pulse sequences oriented in different planes and with different parameter weightings. Each protocol is preprogrammed with the desired parameters. The MRI technician simply calls up the desired protocol from the library to begin scanning.

Preservation of the anatomy surrounding a target surgical site is typically of the utmost importance in any surgery. More specifically, nerve preservation is an important aspect of successful surgeries because it ensures the patient's quality of life is not adversely affected after surgery. Nerve preservation is especially important in pelvic surgery, which may involve the pudendal nerve and its branches as well as erectile nerves such as cavernous nerves.

Although various nerve preservation techniques have been developed, it is often very difficult to determine the precise location of cavernous nerves because of their complicated anatomy and significant variability from one patient to another. For these reasons, the results of nerve-sparing prostatectomies in terms of the preservation of erectile function vary significantly between different clinics and different surgeons and depend heavily on the experience and technique of the surgeon.

In vivo imaging and preoperative precise mapping of erectile nerves such as cavernous nerves can improve their preservation during pelvic surgeries such as, for example, prostate cancer surgery and thus help in preserving a patient's erectile function. Such mapping is particularly challenging since each cavernous nerve is microscopic in diameter (e.g., 100-600 μm) and the number, topology, and location (e.g., ventral, dorsal, or lateral) of cavernous nerves can vary significantly from patient to patient.

A variety of techniques, including electrical and optical nerve stimulation, dye-based optical fluorescence and microscopy, spectroscopy, ultrasound, and MRI have been utilized to identify cavernous nerves and study their anatomy and physiology. Some of these methods may even be utilized intraoperatively to identify and preserve cavernous nerves. However, these methods have proven to be sub-par thus far since the percentage of patients that develop post-surgical erectile disfunction is high.

Given the small size of the objects of cavernous nerves (diameter between about 0.2-0.6 mm), even minimal movements of the internal organs and/or the patient during an MRI can lead to a significant decrease in the quality of the resulting image, which is unacceptable for visualization and mapping of the cavernous nerves.

### SUMMARY

Some exemplary embodiments are related to a method of imaging erectile nerves using a magnetic resonance imaging (MRI) system. The method includes positioning a first radiofrequency (RF) coil on a first side of a body of a patient disposed on an MRI table and a second RF coil on a second side of the body of the patient symmetrical with respect to the first RF coil, wherein the first and second RF coils are positioned proximate a pelvic region of the patient; performing a localizer scan sequence to determine plotting of image slices; performing a T2-weighted true (FISP) cine MRI sequence of the pelvic region to determine whether patient physiological activity corresponds to a desired condition corresponding to a suitability for imaging at least one erectile nerve; generating a plurality of images of the pelvic region using an MRI sequence protocol; and performing post-processing and 3D reconstruction on the plurality of images to map the at least one erectile nerve. Generating the plurality of images of the pelvic region includes performing a T2-weighted turbo spin echo (TSE) MRI sequence of the pelvic region in a sagittal plane; performing a T2-weighted TSE MRI sequence of the pelvic region in an axial plane; performing a T2-weighted TSE MRI sequence of the pelvic region in a coronal plane; performing a T2-weighted TSE high resolution MRI sequence of the pelvic region in the sagittal plane; performing a T2-weighted TSE high resolution MRI sequence of the pelvic region in the axial plane; performing a T2-weighted high resolution MRI sequence of the pelvic region in the coronal plane; performing an isotropic 3-dimensional (3D) fast TSE sequence with fat suppression on the pelvic region; and performing a time-resolved angiography (TWIST_ANGIO) MRI sequence to image blood vessels in the pelvic region.

Some exemplary embodiments are further related to a computer readable storage medium comprising a set of instructions, wherein the set of instructions when executed by a processor cause the processor of a magnetic resonance imaging (MRI) system to perform operations. The operations include performing a localizer scan sequence to determine plotting of image slices; performing a T2-weighted true (FISP) cine MRI sequence of the pelvic region to determine whether patient physiological activity corresponds to a desired condition corresponding to a suitability for imaging at least one erectile nerve; and generating a plurality of images of the pelvic region using an MRI sequence protocol. Generating the plurality of images of the pelvic region includes performing a T2-weighted turbo spin echo (TSE) MRI sequence of the pelvic region in a sagittal plane; performing a T2-weighted TSE MRI sequence of the pelvic region in an axial plane; performing a T2-weighted TSE MRI sequence of the pelvic region in a coronal plane; performing a T2-weighted TSE high resolution MRI sequence of the pelvic region in the sagittal plane; performing a T2-weighted TSE high resolution MRI sequence of the pelvic region in the axial plane; performing a T2-weighted high resolution MRI sequence of the pelvic region in the coronal plane; performing an isotropic 3-dimensional (3D) fast TSE sequence with fat suppression on the pelvic region; and performing a time-resolved angiography (TWIST_ANGIO) MRI sequence to image blood vessels in the pelvic region.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 is a schematic diagram of an exemplary magnetic resonance imaging (MRI) system according to various embodiments.

FIG. 2 shows method of imaging erectile nerves of a patient according to various exemplary embodiments.

FIG. 3A depicts an example of an MRI image taken with surface coils asymmetrically disposed with respect to one another.

FIG. 3B depicts an example of an MRI image taken with surface coils symmetrically disposed with respect to one another.

FIG. 4 shows an exemplary MRI image according to various exemplary embodiments.

FIG. 5 shows an exemplary MRI image according to various exemplary embodiments.

FIG. 6 shows an exemplary MRI image according to various exemplary embodiments.

FIG. 7 shows an exemplary post-processing reconstruction according to various exemplary embodiments.

FIG. 8 shows an exemplary post-processing reconstruction according to various exemplary embodiments.

FIG. 9 shows an exemplary post-processing reconstruction according to various exemplary embodiments.

FIG. 10 shows an exemplary post-processing reconstruction according to various exemplary embodiments.

### DETAILED DESCRIPTION

The exemplary embodiments may be further understood with reference to the following description and the related appended drawings, wherein like elements are provided with the same reference numerals. The exemplary embodiments describe a device, system and method for improved imaging and segmentation of cavernous nerves. The exemplary embodiments are described with regard to a magnetic resonance imaging (MRI) device. In the following description, the phrase “high resolution” with respect to MRI images encompasses images having a spatial resolution between and including 0.1 mm×0.1 mm×1.0 mm-0.3 mm×0.3 mm×3.0 mm.

As noted above, conventional imaging techniques used to obtain images of erectile nerves such as, for example, cavernous nerves have often yielded unsatisfactory results as, due to their small size, any organ and/or patient motion during imaging adversely affects image quality of the cavernous nerves.

According to exemplary embodiments, a method for imaging erectile nerves includes predetermined patient placement and scan protocol that substantially eliminates or reduces motion artifacts. As a result of the improved imaging of the erectile nerves, the probability of sparing these nerves during prostate surgery is significantly improved.

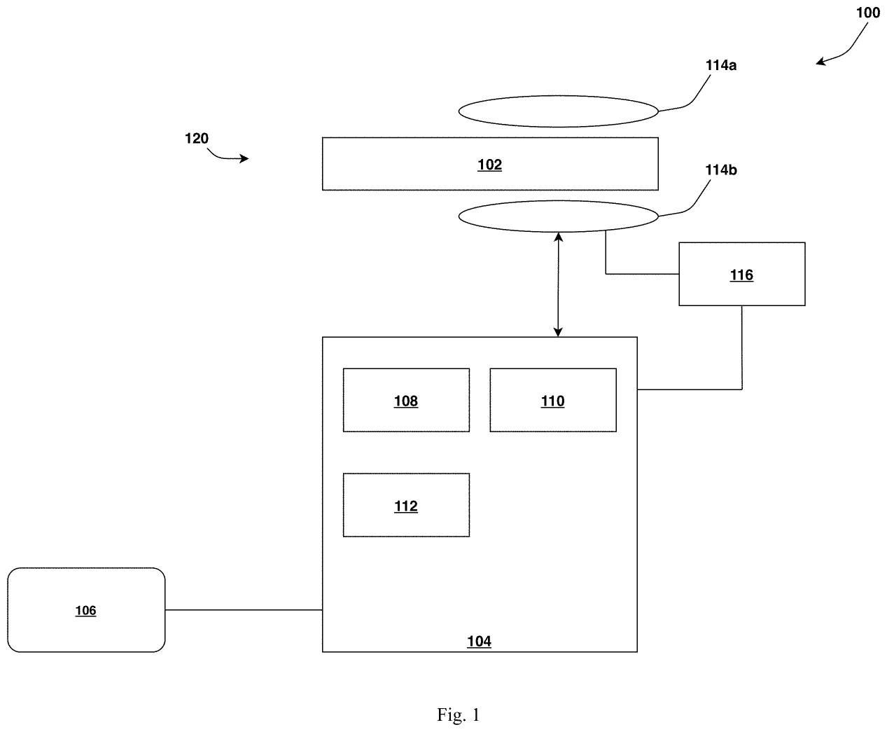

FIG. 1 is a schematic diagram of an exemplary magnetic resonance imaging (MRI) system 100 according to various embodiments. The imaging system includes a computing device 104 which, as would be understood by those skilled in the art, may represent any suitable electronic computing device. The computing device 104 includes a display device 106, a processor 108, a memory arrangement 110, and an input/output (I/O) device 112. The MRI system 100 further includes an MRI device 120 communicatively coupled to the computing device 104.

The MRI device 120 includes a table 102, a first radiofrequency (RD) coil 114a disposed above a table 102, a second RF coil 114b disposed below the table 102, an RF excitation device coupled to the RF coils 114a, 114b, and an MR detection apparatus configured to detect signals after they have engaged target tissue within the body of a patient. It should be noted that this description and illustration is merely illustrative and that the MRI system 100 may include additional or alternative components to facilitate the method discussed below.

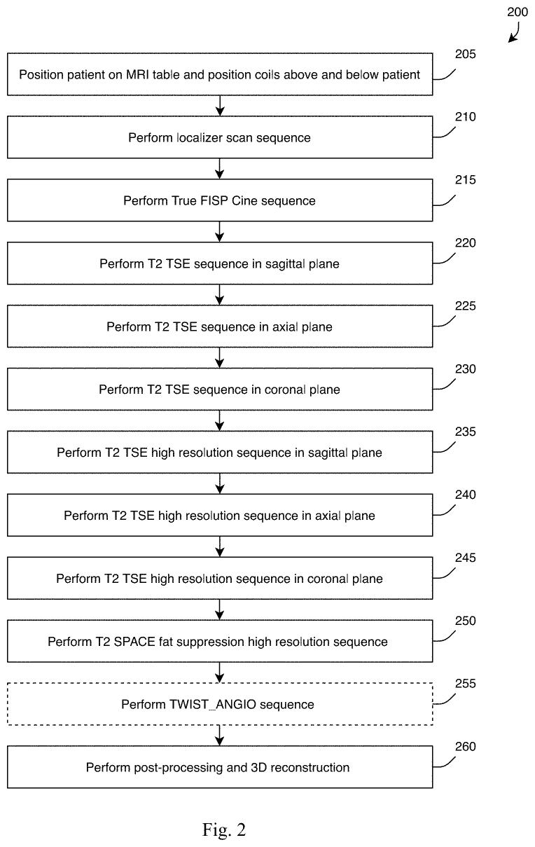

FIG. 2 shows a method 200 of imaging erectile nerves of a patient according to various exemplary embodiments. It should be noted that prior to imaging, the patient is generally instructed to follow a predetermined diet for a predetermined time period prior to the day of imaging, e.g., to reduce flatulence on the day of imaging. In addition, the patient may be instructed to take prescription medication such as, for example, simethicone, for two or more days prior to the day of imaging. In some embodiments, the prescription is to take two capsules of 40 mg of simethicone three times per day for two days. Those skilled in the art will understand that it is desirable to select a patient regimen that will reduce or eliminate flatulence and peristaltic movement during imaging to minimize or eliminate movement of adjacent bodily structures to enhance the clarity of the imaging of the target nerves and that other combinations of diet and/or prescription regimen may also be used to achieve this aim.

In addition to the preparation of the patient during the days leading up to imaging, a predetermined drug regimen may be given to the patient on the day the imaging is performed. In some embodiments, the predetermined drug regimen is defined in Table 1 below. This drug regimen is aimed at reducing flatulence during the imagine process, which advantageously reduces motion artifacts in the resulting images. The effect of the predetermined drug regimen additionally reduces peristaltic movement of the intestines for 2-3 hours, which further reduces motion artifacts in the resulting images.

Microlax® cleanses the patient's intestines from feces, thus preventing artifacts from feces appearing in the MRI images. Buscopan® exerts a spasmolytic action on the smooth muscle of the gastrointestinal, biliary, and genito-urinary tracts, thus minimizing the peristalsis (automatic contractions) of the intestine. As a result, movement artifacts caused by the intestines are avoided during MRI scanning. Espumisan® is an anti-foaming agent that decreases the surface tension of gas bubbles, causing them to combine into larger bubbles in the digestive tract, which prevents gas and/or flatulence. Loperamide® inhibits gut motility by binding to opiate receptors in the gut wall and may also reduce gastrointestinal secretions. The combination of these drugs ensures that artifacts that may arise during imaging are significantly reduced or eliminated.

TABLE 1

| | 2 hours | 1 hour | 15 minutes | Immediately |
|---|---|---|---|---|
| | before | before | before | before |
| Drug | MRI | MRI | MRI | MRI |
| MICROLAX ® | 1 enema | | | |
| (Sodium citrate, | | | | |
| Sorbitol) | | | | |
| BUSCOPAN ® 10 mg | 5 tab. | 5 tab. | | 5 tab. |
| (Hyoscine | | | | |
| butylbromide) | | | | |
| ESPUMISAN ® 40 mg | 2 caps. | | | |
| (Simethicone) | | | | |
| LOPERAMIDE ® | | | 2 tab. | 1 tab. |
| (Loperamide | | | | |
| hydrochloride) | | | | |
| 2 mg | | | | |

After the patient has been properly prepared, as outlined above, the method proceeds to 205, where the patient is placed on the MRI table 102 and the upper and lower RF coils 114a,b are placed above and below the patient, respectively. The patient is placed on the MRI table 102 with their legs or head forward, depending on the location of the connectors that couple to the RF coils (i.e., the connectors that couple the RF source to the coils). A multi-channel (at least 18 channels) RF coil (RF coil 114b) is placed beneath the patient at the pelvic area. The patient is placed on the coil in such a way that the zone of interest is located at the center of the RF coil. To determine proper patient positioning, for example, a large trochanter of each femur is palpated from both sides to ensure that it is at the proportional middle of the RF coil left and right.

A similar RF coil (RF coil 114a) is placed on top of the patient so that its scanning elements are located substantially symmetrically relative to the scanning elements of the lower RF coil 114b. In some embodiments, the upper RF coil 114a coil may be fixed with seat belts to the MRI table 102. The force of contraction by the seat belts on the upper RF coil 114a should prevent the coil from moving during scanning but should also not create discomfort to the patient. In some embodiments, spacers may be placed between the coils to minimize deformation of the upper RF coil 114a.

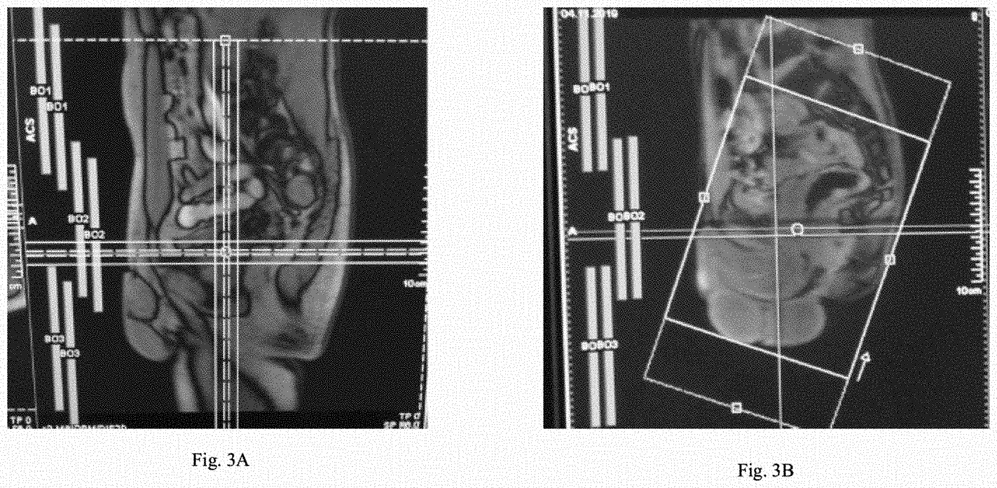

In routine practice using current techniques, strictly symmetrical placement of the upper and lower coils is often not employed. With an asymmetric placement of the coils, segments of the resulting images are shifted. As a result, the final reconstruction is averaged, reducing the clarity of small structures. An example of this scenario is depicted in FIG. 3A, which depicts an MRI image taken with asymmetrically placed coils. As illustrated in the localizer image of FIG. 3A, the coils (labelled B03) are asymmetrical with respect to one another, which reduces image quality. In contrast, the localizer image of FIG. 3B depicts an example of an MRI image taken with symmetric coils (also labelled B03). The resulting image exhibits a more accurate image segment acquisition by each coil element and, as a result, image reconstruction is clearer. As such, in the present embodiments, the coils 114a, 114b are preferably positioned symmetric to one another in 205 to minimize motion artifacts and obtain a clearer reconstruction.

To determine the symmetry of the RF coils 114a, 114b, a function of the MRI system 100 that allows for the display of the scanning elements of the RF coils 114a,b on the display device 106 is activated. A localizer sequence is performed and, subsequently, a second localizer sequence is added to visually evaluate the location of the scanning elements on the display device 106.

After the symmetric coil placement has been verified, the MRI scan protocol (210-255) commences. At 210, a localizer scan sequence is performed using the MRI system 100 in three perpendicular planes to mark subsequent sequences. The resulting localizer images are used for plotting slices. At 215 a true fast imaging with steady-state precession (True FISP) cine sequence is conducted. This sequence is a dynamic T2 weighted sequence performed in the sagittal plane in the zone of interest (e.g., pelvic area) with one slice with a plurality of repetitions.

In some embodiments, the number of repetitions may be between about 30 to about 80 depending on the data collection time. The data collection time should not exceed 2 seconds, with the total duration being at least 60 seconds. Using this sequence, the preparation of the patient, the period of peristaltic movements, and the volume of the patient's bladder are evaluated. Based on these findings, a determination is made as to whether subsequent scanning may commence or whether re-preparation of the patient is required.

It should be noted that True FISP cine protocol (and other similar protocols) are not typically used in current processes to evaluate the quality of patient preparation. However, in the present disclosure, patient preparation is important to minimize movement artifacts (both of the patient and the patient's internal organs) and to maximize the quality and clarity of the resulting images.

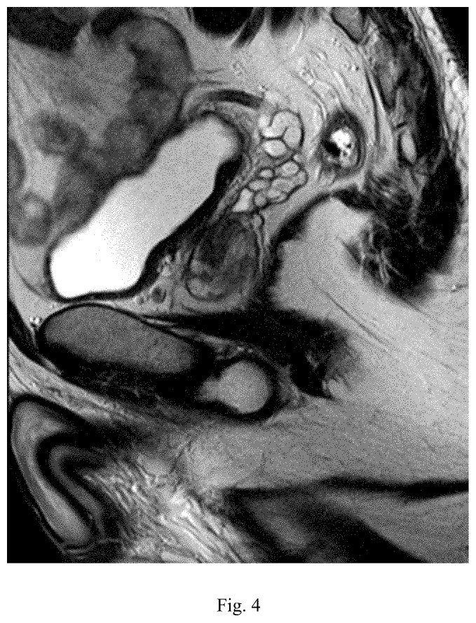

At 220, a 3.5 mm T2-weighted turbo spin echo (TSE) (or fast spin echo depending on the brand MRI system) MRI sequence is performed on the target area (the pelvic region) in the sagittal plane. In addition to a slice thickness of, for example, 3.5 mm, the number of slices for this sequence is set so that the entire target area is covered. TR and TE are adjusted until a desired contrast of soft tissue is achieved. An example of a resulting image from this sequence is shown in FIG. 4.

In some embodiments, the TR is between about 2200 ms and about 3500 ms and the TE is between about 60 ms and about 90 ms. In some embodiments, the TR is between about 2400 ms and about 2600 ms and the TE is between about 70 ms and about 80 ms. In some embodiments, the TR is about 2580 ms and the TE is about 77 ms. The optimal contrast will vary from patient to patient but should provide good contrast of the prostate, seminal vesicles, bladder, rectum, and cavernous and spongious bodies of the penis with clear boundaries of these organs.

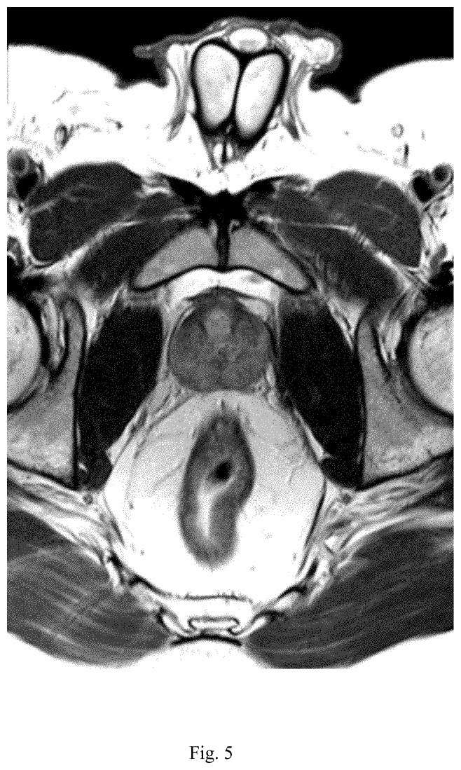

At 225, a 3.5 mm T2-weighted TSE MRI sequence is performed on the target area in the axial plane. In addition to a slice thickness of 3.5 mm, the number of slices for this sequence is preferably set so that the entire target area is covered. TR and TE are adjusted until a desired contrast of soft tissue is achieved. An example of a resulting image from this sequence is shown in FIG. 5. In some embodiments, the TR is between about 2200 ms and about 3500 ms and the TE is between about 60 ms and about 90 ms. In some embodiments, the TR is between about 2400 ms and about 2600 ms and the TE is between about 70 ms and about 80 ms. In some embodiments, the TR is about 2580 ms and the TE is about 77 ms. The optimal contrast will vary from patient to patient but should provide good contrast of the prostate, seminal vesicles, bladder, rectum, and cavernous and spongious bodies of the penis with clear boundaries of these organs.

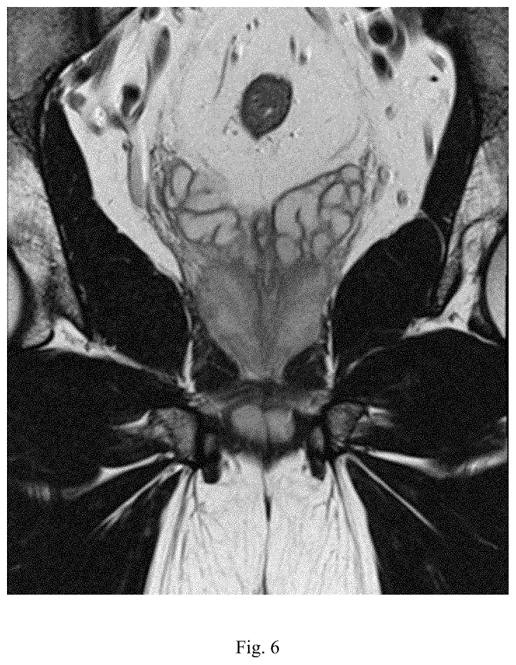

At 230, a 3.5 mm T2-weighted TSE MRI sequence is performed on the target area in the coronal plane. In addition to a slice thickness of, for example, 3.5 mm, the number of slices for this sequence is set in these exemplary embodiments so that the entire target area is covered. TR and TE are adjusted until a desired contrast of soft tissue is achieved. An example of a resulting image from this sequence is shown in FIG. 6. In some embodiments, the TR is between about 2200 ms and about 3500 ms and the TE is between about 60 ms and about 90 ms.

In some embodiments, the TR is between about 2400 ms and about 2600 ms and the TE is between about 70 ms and about 80 ms. In some embodiments, the TR is about 2580 ms and the TE is about 77 ms. The optimal contrast will vary from patient to patient but should provide good contrast of the prostate, seminal vesicles, bladder, rectum, and cavernous and spongious bodies of the penis with clear boundaries of these organs.

At 235, a high resolution T2-weighted TSE 2D MRI sequence is performed on the target area in the sagittal plane. This TSE sequence is used to visualize anatomical structures in the sagittal plane. The TR is adjusted so that, in one embodiment it is between about 2200 ms and about 3500 ms. In some embodiments, the TR is between about 2400 ms and about 2600 ms. In some embodiments, the TR is about 2580 ms. The TE is adjusted so that it is between about 60 ms and about 90 ms. In some embodiments, the TE is between about 70 ms and about 80 ms. In some embodiments, the TE is about 77 ms.

In some embodiments, the minimum spatial resolution of the resulting tomogram is 0.3 mm×0.3 mm×3.0 mm. In some embodiments, the minimum spatial resolution of the resulting tomogram is 0.2 mm×0.2 mm×2.0 mm. In some embodiments, the minimum spatial resolution of the resulting tomogram is 0.1 mm×0.1 mm×1.0 mm. The resulting tomogram provides improved clarity compared with current pelvic imaging (e.g., provided by the PI-RADS system), which has a spatial resolution of 0.6 mm×0.6 mm×3.0 mm.

Additional parameter settings of the high resolution T2-weighted TSE 2D MRI sequence are (1) slice gap of 0 mm, (2) a turbo factor between about 11 and about 17, (3) a flip angle between 140° and about 180°, (4) “restore magnetization” is toggled on, (5) the field of view is less than or equal to 190×160 mm, (6) the number of repetitions of data acquisition is between about 8 and about 21, (7) the pixel bandwidth is greater than or equal to about 250 Hz per pixel, and (8) the RF pulse is applied in Fast, Normal, or Low specific absorption rate (SAR). In some embodiments, the RF pulse is applied in the Fast mode.

In some embodiments, fat suppression techniques may be applied to ensure the signal from fat tissue is suppressed, thus resulting in improved nerve visualization. Such fat suppression techniques may include, for example, frequency-selective fat suppression, short T1 inversion recovery (STIR) with T1 inversion between about 160 ms and about 180 ms on a 1.5 T MRI scanner or between about 200 ms and about 240 ms on a 3.0 T MRI scanner, or spectral attenuated inversion recovery (SPAIR).

Since slice gaps would add a space between the slices, a slice gap of 0 ensures that tiny structures such as, for example, cavernous nerves are not missed. The turbo factor is the number of echoes acquired after each excitation. This is a measure of the scan time acceleration. The TSE turbo factor, together with the effective TE, controls the echo spacing, which is the temporal distance between the echoes in multiple echo sequences (e.g., echo planar imaging, fast spin echo).

A short echo space produces compact sequence timing and fewer image artifacts. The shorter the rise time, the faster the gradients and, therefore, the echo spacing. Gradients with a shorter echo spacing will have a higher resolution and more slices per TR. As such, the turbo factor affects the tissue contrast. The flip angle is the amount of rotation the net magnetization experiences during application of a radiofrequency pulse. This affects patient heating during scanning. Toggling the restore magnetization on prevents oversaturation of the tissues with energy. The field of view (FOV) is the distance over which an MR image is acquired or displayed. The FOV is typically divided into several hundred picture elements (pixels), each having a size of about 1 mm².

The optimal number of repetitions depends on the type of MRI scanner, gradients, and surface coils. The number of repetitions affects the signal-to-noise ratio (and, consequently, the image quality). The higher the initial signal-to-noise ratio, the fewer number of repetitions are needed. Increasing the receiver bandwidth can be used to produce less blurry images because it reduces the echo spacing. In TSE sequences, higher bandwidths increase the turbo factor due to short echo spacing.

To reduce motion artifacts, the phase encoding direction is set to anterior-posterior, the phase encoding vector is directed at an angle between about 70° and about 90° to the plane of the MRI table 102, and a parallel imaging technique (e.g., iPAT from Siemens, GRAPPA, CAIPIRINHA, or SyncraScan from Siemans, or ASSET, GEM, ARC from General Electric) is used to reduce scan time. The phase encoding direction is set to anterior-posterior because this is the usual direction of patient's movements and abdominal wall movements during breathing. As such, the anterior-posterior phase encoding setting reduces motion artifacts in the resulting images.

The number of repetitions of data acquisition is a multiple of the factor of parallelization of data acquisition. To reduce flow artifacts, the mode of suppressing flow artifacts in the direction of the phase or slice encoding vector is turned on, depending on the direction of the distribution of the artifact. Flow artifacts are caused by blood flow or fluid flow in the body. Liquid flowing through a slice may experience an RF pulse and then flow out of the slice before the signal is recorded. Thus, it is beneficial to use modes of suppressing flow artifacts to minimize or eliminate this issue. To comply with TR limits and avoid using concatenation, which reduces image quality, slices may be grouped into several blocks. For example, slices may be grouped as (a) left and right or (b) left, center, and right. The grouped blocks are subsequently combined into a single block.

AT 240, a high resolution T2-weighted TSE 2D MRI sequence is performed on the target area in the axial plane. This TSE sequence is used to visualize anatomical structures in the axial plane. The TR is adjusted so that it is between about 2200 ms and about 3500 ms. In some embodiments, the TR is between about 2400 ms and about 2600 ms. In some embodiments, the TR is about 2580 ms. The TE is adjusted so that it is between about 60 ms and about 90 ms. In some embodiments, the TE is between about 70 ms and about 80 ms. In some embodiments, the TE is about 77 ms.

In some embodiments, the minimum spatial resolution of the resulting tomogram is 0.3 mm×0.3 mm×3.0 mm. In some embodiments, the minimum spatial resolution of the resulting tomogram is 0.2 mm×0.2 mm×2.0 mm. In some embodiments, the minimum spatial resolution of the resulting tomogram is 0.1 mm×0.1 mm×1.0 mm. The resulting tomogram provides improved clarity compared with current pelvic imaging (e.g., provided by the PI-RADS system), which has a spatial resolution of 0.6 mm×0.6 mm×3.0 mm.

Additional parameter settings of the high resolution T2-weighted TSE 2D MRI sequence are (1) slice gap of 0 mm, (2) a turbo factor between about 11 and about 17, (3) a flip angle between 140° and about 180°, (4) “restore magnetization” is toggled on, (5) the field of view is less than or equal to 190×160 mm, (6) the number of repetitions of data acquisition is between about 8 and about 21, (7) the pixel bandwidth is greater than or equal to about 250 Hz per pixel, and (8) the RF pulse is applied in Fast, Normal, or Low SAR. In some embodiments, the RF pulse is applied in the Fast mode.

To reduce motion artifacts, the phase encoding direction is set to anterior-posterior or right-left, the plane of the slices is perpendicular to the sagittal plane, and a parallel imaging technique is used. The number of repetitions of data acquisition is a multiple of the factor of parallelization of data acquisition. To reduce flow artifacts, the mode of suppressing flow artifacts in the direction of the phase or slice encoding vector is turned on, depending on the direction of the distribution of the artifact. To comply with TR limits and avoid using concatenation, which reduces image quality, slices may be grouped into several blocks. For example, slices may be grouped as (a) left and right or (b) left, center, and right. The grouped blocks are subsequently combined into a single block.

At 245, a high resolution T2-weighted TSE 2D MRI sequence is performed on the target area in the coronal plane. This TSE sequence is used to visualize anatomical structures in the coronal plane. The TR is adjusted so that it is between about 2200 ms and about 3500 ms. In some embodiments, the TR is between about 2400 ms and about 2600 ms. In some embodiments, the TR is about 2580 ms. The TE is adjusted so that it is between about 60 ms and about 90 ms. In some embodiments, the TE is between about 70 ms and about 80 ms. In some embodiments, the TE is about 77 ms.

In some embodiments, the minimum spatial resolution of the resulting tomogram is 0.3 mm×0.3 mm×3.0 mm. In some embodiments, the minimum spatial resolution of the resulting tomogram is 0.2 mm×0.2 mm×2.0 mm. In some embodiments, the minimum spatial resolution of the resulting tomogram is 0.1 mm×0.1 mm×1.0 mm. The resulting tomogram provides improved clarity compared with current pelvic imaging (e.g., provided by the PI-RADS system), which has a spatial resolution of 0.6 mm×0.6 mm×3.0 mm.

Additional parameter settings of the high resolution T2-weighted TSE 2D MRI sequence are, in this exemplary embodiment, (1) slice gap of 0 mm, (2) a turbo factor between about 11 and about 17, (3) a flip angle between 140° and about 180°, (4) “restore magnetization” is toggled on, (5) the field of view is less than or equal to 190×160 mm, (6) the number of repetitions of data acquisition is between about 8 and about 21, (7) the pixel bandwidth is greater than or equal to about 250 Hz per pixel, and (8) the RF pulse is applied in Fast, Normal, or Low SAR. In some embodiments, the RF pulse is applied in the Fast mode.

To reduce motion artifacts, the phase encoding direction is set to head-feet or right-left, the plane of the slices is perpendicular to the sagittal plane, and a parallel imaging technique is used. The number of repetitions of data acquisition is a multiple of the factor of parallelization of data acquisition. To reduce flow artifacts, the mode of suppressing flow artifacts in the direction of the phase or slice encoding vector is turned on, depending on the direction of the distribution of the artifact. To comply with TR limits and avoid using concatenation, which reduces image quality, slices may be grouped into several blocks. For example, slices may be grouped as (a) left and right or (b) left, center, and right. The grouped blocks are subsequently combined into a single block.

At 250, a T2-weighted single slab 3D TSE sequence (e.g., T2_SPACE_FS) with a slab selective, variable excitation pulse having a fat-suppression technique (e.g., frequency-selective fat suppression, STIR, SPAIR) with a weighting close to a fat-suppressed T2 weighted sequence. This sequence enables acquisition of high-resolution 3D datasets with contrasts similar to those obtained from 2D T2-weighted, T1-weighted, proton density and dark fluid protocols. This sequence is used to visualize nerves and blood vessels (e.g., cavernous nerves), lymph nodes, and ducts. Data is collected in the axial, sagittal, or coronal planes. In some embodiments, the data is collected in the axial plane.

Parameter settings of the T2-weighted single slab 3D TSE sequence are (1) MR acquisition type is set to 3D, (2) the TR is between about 900 ms and about 1100 ms, and (3) the TE is between about 70 ms and about 160 ms. The minimal spatial resolution of the resulting tomogram is 0.6 mm×0.6 mm 0.6 mm. In some embodiments, the minimal spatial resolution of the resulting tomogram is 0.5 mm×0.5 mm 0.5 mm. In some embodiments, the minimal spatial resolution of the resulting tomogram is 0.3 mm×0.3 mm 0.3 mm. In some embodiments, the minimal spatial resolution of the resulting tomogram is 0.1 mm×0.1 mm 0.1 mm.

Further parameter settings for the T2-weighted single slab 3D TSE sequence are (1) slice gap of 0 mm, (2) a turbo factor between about 40 and about 100, (3) a flip angle between 140° and about 180°, (4) the RF pulse is applied in Fast, Normal, or Low SAR, (5) “restore magnetization” is toggled off, (6) the field of view is greater than or equal to 280×280 mm, (7) the number of repetitions of data acquisition is between about 1.4 and about 4, (8) the pixel bandwidth is greater than or equal to about 700 Hz per pixel, and (9) a fat-suppression technique is applied. Examples of fat suppression techniques are frequency-selective fat suppression, STIR (with T1 between about 160 ms and about 180 ms on a 1.5 T MRI system or about 200 ms and about 240 ms on a 3 T MRI system), or SPAIR.

In some embodiments, at 255, a time-resolved angiography with interleaved stochastic trajectories (TWIST_ANGIO) sequence may optionally be used to image blood vessels (prostatic and other adjustment vessels) in the target area. The TWIST_ANGIO is a time resolved 3D magnetic resonance angiography (MRA) with a high temporal resolution with a gadolinium-based contrast agent administration.

The parameters for the TWIST_ANGIO are (1) TR and TE set at their minimum settings, (2) a flip angle between about 20° and about 30°, and (3) data acquisition speed between about 1 second and about 6 seconds. In some embodiments, the minimum spatial resolution of the resulting tomogram is 1.0 mm×1.0 mm×2.0 mm. In some embodiments, the minimum spatial resolution of the resulting tomogram is 0.5 mm×0.5 mm×1.0 mm. Additional parameters for the TWIST_ANGIO are (1) the number of repetitions is 1, (2) the number of phases is between about 10 and about 15, (3) the pixel bandwidth is greater than or equal to about 1000 Hz per pixel, (4) the total data acquisition time is between about 180 seconds and about 300 seconds, and (5) the contrast agent administration is about 3.5 ml/sec.

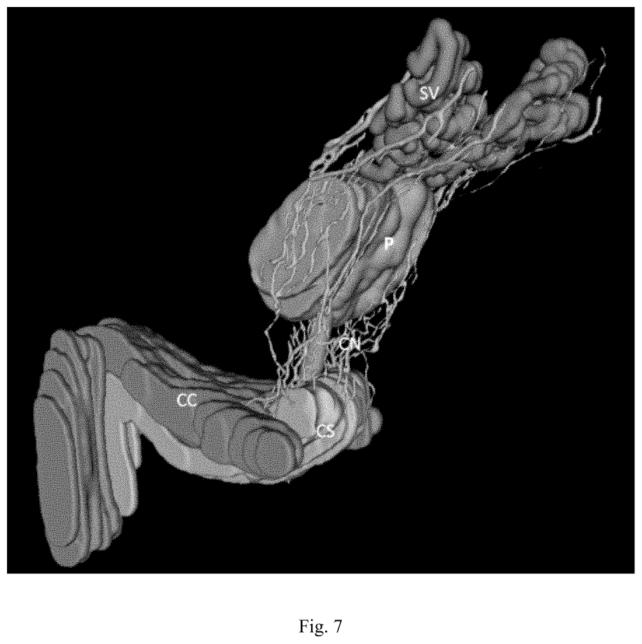

At 260, post-processing and 3D reconstruction of the differentiated nerves is performed using, for example, software having 3D segmentation wide opportunities such as, for example, 3D SLICER®, Inobitec DICOM Viewer, etc. This is illustrated in FIG. 7, where CN depicts the cavernous nerves, SV depicts the seminal vesicles, P depicts the prostate, CS depicts the corpus spongiosum, and CC depicts the corpus cavernosum. The purpose of this modelling is to map erectile nerves (both cavernous and pudendal) and vessels around the prostate down to the crus of the penis.

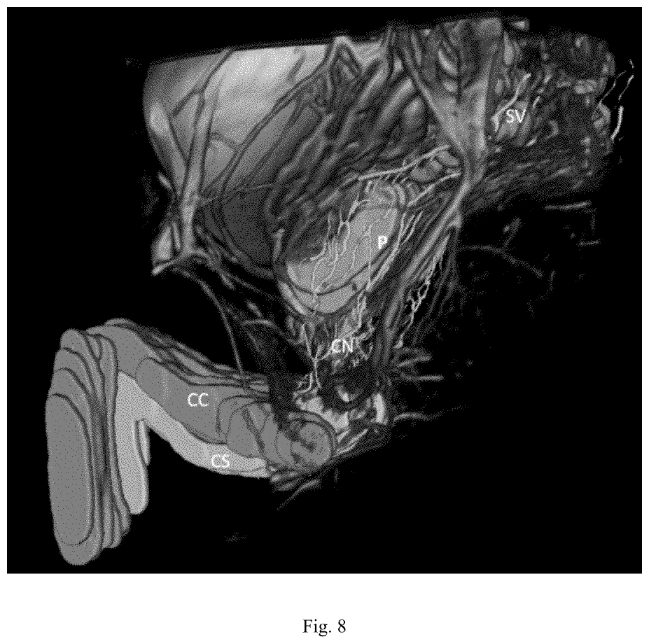

In addition, the main pelvic organs (e.g., the prostate, seminal vesicles, urethra, rectum, cavernous and spongious bodies of the penis, etc.) are segmented for 3D modelling. During mapping of the target nerves, T2-weighted high-resolution images are fused (superimposed) with the T2-weighted single slab 3D TSE high resolution images (e.g., T2 SPACE FS) and/or TWIST_ANGIO images to help differentiate nerves and vessels and to improve the accuracy of nerve segmentation. In addition, nerves are tracked and matched in at least two mutually perpendicular planes. This is shown in FIGS. 8-10.

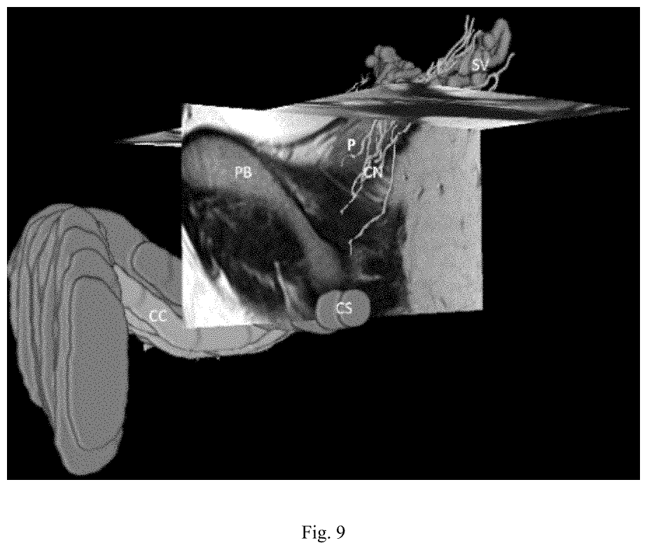

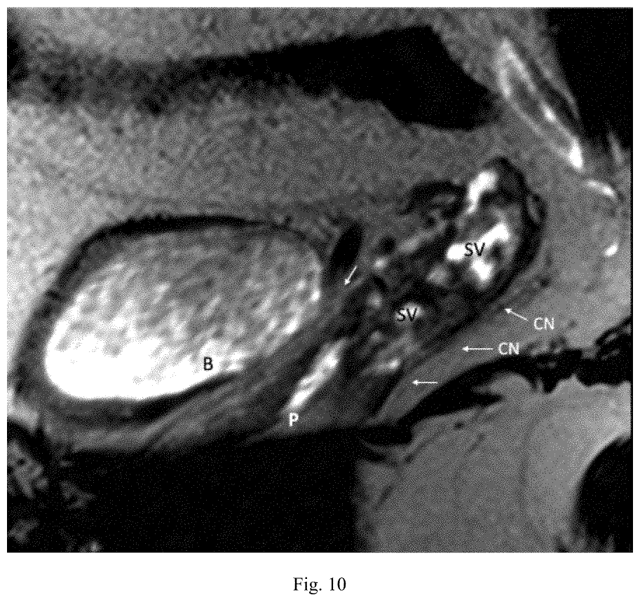

In FIG. 8, a 3D SLICER® MRI-based model of the cavernous nerves is fused with the T2-weighted single slab 3D TSE high resolution images (e.g., T2_space_FS model) with vessel segmentation. In FIG. 9, a 3D SLICER® MRI-based model of the cavernous nerves is fused with native T2-weighted TSE axial and sagittal images. FIG. 10 depicts cavernous nerve mapping where the T2-weighted TSE sagittal image is fused with the T2-weighted single slab 3D TSE high resolution image showing bright, fluid liquid content structures. This helps to differentiate nerves and vessels.

Those skilled in the art will understand that the above-described exemplary embodiments may be implemented in any suitable software or hardware configuration or combination thereof. In a further example, the exemplary embodiments of the above described method may be embodied as a program containing lines of code stored on a non-transitory computer readable storage medium that, when compiled, may be executed on a processor or microprocessor. For example, as explained above, most MRI systems are preprogrammed with the desired parameters for a procedure. In some embodiments, the MRI system 100 may be preprogrammed so that an MRI technician can just call up the protocol and the processor 108 of the computing device 104 will execute instructions stored on the memory arrangement 110 to perform the method 200.

Although this application described various aspects each having different features in various combinations, those skilled in the art will understand that any of the features of one aspect may be combined with the features of the other aspects in any manner not specifically disclaimed or which is not functionally or logically inconsistent with the operation of the device or the stated functions of the disclosed aspects.

It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

It will be apparent to those skilled in the art that various modifications may be made in the present disclosure, without departing from the spirit or the scope of the disclosure. For example, use of the term “about” when discussing specific values means+ or −10% of the disclosed parameter values. Thus, it is intended that the present disclosure cover modifications and variations of this disclosure provided they come within the scope of the appended claims and their equivalent.

What is claimed:

1. A method for imaging erectile nerves using a magnetic resonance imaging (MRI) system, comprising:
positioning a first radiofrequency (RF) coil on a first side of a body of a patient disposed on an MRI table and a second RF coil on a second side of the body of the patient symmetrical with respect to the first RF coil, wherein the first and second RF coils are positioned proximate a pelvic region of the patient;
performing a localizer scan sequence to determine plotting of image slices;
performing a T2-weighted true (FISP) cine MRI sequence of the pelvic region to determine whether patient physiological activity corresponds to a desired condition corresponding to a suitability for imaging at least one erectile nerve;
generating a plurality of images of the pelvic region using an MRI sequence protocol comprising:
performing a T2-weighted turbo spin echo (TSE) MRI sequence of the pelvic region in a sagittal plane;
performing a T2-weighted TSE MRI sequence of the pelvic region in an axial plane;
performing a T2-weighted TSE MRI sequence of the pelvic region in a coronal plane;
performing a T2-weighted TSE high resolution MRI sequence of the pelvic region in the sagittal plane;
performing a T2-weighted TSE high resolution MRI sequence of the pelvic region in the axial plane;
performing a T2-weighted high resolution MRI sequence of the pelvic region in the coronal plane;
performing an isotropic 3-dimensional (3D) fast TSE sequence with fat suppression on the pelvic region; and
performing a time-resolved angiography (TWIST_ANGIO) MRI sequence to image blood vessels in the pelvic region; and
performing post-processing and 3D reconstruction on the plurality of images to map the at least one erectile nerve.

2. The method of claim 1, wherein the localizer scan sequence is performed in three perpendicular planes.

3. The method of claim 1, wherein the T2-weighted true FISP cine sequence is performed in the sagittal plane with one slice for a plurality of repetitions, wherein collection of one data element does not exceed 2 seconds, and wherein a total duration of the T2-weighted true FISP cine sequence is at least 60 seconds.

4. The method of claim 1, wherein each of (a) the T2-weighted TSE high resolution MRI sequence of the pelvic region in the sagittal plane, (b) the T2-weighted TSE high resolution MRI sequence of the pelvic region in the axial plane, and (c) the T2-weighted TSE high resolution MRI sequence of the pelvic region in the coronal plane includes a predetermined set of parameters comprising:
a repetition time (TR) between 2200 ms and 3500 ms;
an echo time (TE) between 60 ms and 90 ms;
a slice gap of 0 mm;
a turbo factor between 11 and 17;
a flip angle between 140° and 180°;
an RF pulse applied in a fast mode, a normal mode, or a low specific absorption rate (SAR) mode;
activation of a restore magnetization parameter;
a field of view no greater than 190 mm×160 mm;
a number of repetitions of data acquisition between 8 and 21; and
a pixel bandwidth no less than to 250 Hz per pixel,
wherein a minimum spatial resolution of a resulting tomogram is 0.3 mm×0.3 mm×3.0 mm.

5. The method of claim 4, wherein the T2-weighted TSE high resolution MRI sequence of the pelvic region in the sagittal plane includes a second set of parameters configured to reduce motion artifacts, the second set of parameters comprising:
a phase encoding direction set to anterior-posterior;
a phase encoding vector directed at an angle between 70 and 90 to the MRI table;
a parallel imaging technique; and
a number of repetitions equal to a multiple of a factor of parallelization of data acquisition.

6. The method of claim 4, wherein the T2-weighted TSE high resolution MRI sequence of the pelvic region in the axial plane includes a second set of parameters configured to reduce motion artifacts, wherein the second set of parameters comprises:
a phase encoding direction set to anterior-posterior or right-left;
a phase encoding vector perpendicular to the sagittal plane;
a parallel imaging technique; and
a number of repetitions equal to a multiple of a factor of parallelization of data acquisition.

7. The method of claim 4, wherein the T2-weighted TSE high resolution MRI sequence of the pelvic region in the coronal plane includes a second set of parameters configured to reduce motion artifacts, the second set of parameters comprising:
a phase encoding direction set to head-feet or right-left;
a plane of slices perpendicular to the sagittal plane;
a parallel imaging technique; and
a number of repetitions equal to a multiple of a factor of parallelization of data acquisition.

8. The method of claim 4, wherein the TR is between 2400 ms and 2600 ms.

9. The method of claim 8, wherein the TR is 2580 ms.

10. The method of claim 4, wherein the TE is between 70 ms and 80 ms.

11. The method of claim 10, wherein the TE is 77 ms.

12. The method of claim 4, wherein the RF pulse is applied in the fast mode.

13. The method of claim 4, wherein the minimum spatial resolution of the resulting tomogram is 0.2 mm×0.2 mm×2.0 mm.

14. The method of claim 13, wherein the minimum spatial resolution of the resulting tomogram is 0.1 mm×0.1 mm×1.0 mm.

15. The method of claim 1, wherein the 3D fat suppression MRI sequence on the pelvic region includes a predetermined set of parameters comprising:
a TR between 900 ms and 1100 ms;
a TE between 70 ms and 160 ms;
a slice gap of 0 mm;
a turbo factor between 40 and 100;
a flip angle between 140° and 180°;
an RF pulse applied in a fast mode, a normal mode, or a low SAR mode;
deactivation of a restore magnetization parameter;
a field of view no less than 280 mm×280 mm;
a number of repetitions of data acquisition between 1.4 and about 4; and
a pixel bandwidth greater than or equal to 700 Hz per pixel,
wherein a minimum spatial resolution of a resulting tomogram is 0.6 mm×0.6 mm×0.6 mm.

16. The method of claim 15, wherein the minimum spatial resolution of the resulting tomogram is 0.5 mm×0.5 mm×0.5 mm.

17. The method of claim 15, wherein the minimum spatial resolution of the resulting tomogram is 0.3 mm×0.3 mm×0.3 mm.

18. The method of claim 15, wherein the minimum spatial resolution of the resulting tomogram is 0.1 mm×0.1 mm×0.1 mm.

19. The method of claim 1, wherein the 3D fat suppression MRI sequence on the pelvic region is performed in the axial plane.

20. The method of claim 1, wherein the TWIST_ANGIO MRI sequence includes a predetermined set of parameters comprising:
a TR set to a minimum TR setting;
a TE set to a minimum TE setting;
a flip angle between 20° and 30°;
a data acquisition speed between 1 second and 6 seconds;
a single repetition of data acquisition;
a phase number between 10 and 15;
a pixel bandwidth no less than 1000 Hz per pixel; and
a total data acquisition time between 180 seconds and 300 seconds,
wherein a minimum spatial resolution of a resulting tomogram is 1.0 mm×1.0 mm×2.0 mm.

21. A computer readable storage medium comprising a set of instructions, wherein the set of instructions when executed by a processor cause the processor of a magnetic resonance imaging (MRI) system to perform operations, comprising:
performing a localizer scan sequence to determine plotting of image slices;
performing a T2-weighted true (FISP) cine MRI sequence of the pelvic region to determine whether patient physiological activity corresponds to a desired condition corresponding to a suitability for imaging at least one erectile nerve; and
generating a plurality of images of the pelvic region using an MRI sequence protocol comprising:
performing a T2-weighted turbo spin echo (TSE) MRI sequence of the pelvic region in a sagittal plane;
performing a T2-weighted TSE MRI sequence of the pelvic region in an axial plane;
performing a T2-weighted TSE MRI sequence of the pelvic region in a coronal plane;
performing a T2-weighted TSE high resolution MRI sequence of the pelvic region in the sagittal plane;
performing a T2-weighted TSE high resolution MRI sequence of the pelvic region in the axial plane;
performing a T2-weighted high resolution MRI sequence of the pelvic region in the coronal plane;
performing an isotropic 3-dimensional (3D) fast TSE sequence with fat suppression on the pelvic region; and
performing a time-resolved angiography (TWIST_ANGIO) MRI sequence to image blood vessels in the pelvic region.
